(19) United States
(12) Patent Application Publication
(10) Pub. No.: US 2006/0122498 A1
(43) Pub. Date: Jun. 8, 2006

(54) Optical projection tomography

(76) Inventor: James Alexander Sharpe, Edinburgh (GB)

Correspondence Address: BARNES & THORNBURG, LLP, P.O. BOX 2786, CHICAGO, IL, 60690-2786, US

(21) Appl. No.: 10/522,932

(22) PCT Filed: Aug. 29, 2003
(86) PCT No.: PCT/GB03/03726

§ 371 Date: Sep. 26, 2005

(30) Foreign Application Priority Data
Aug. 30, 2002 (GB) 0220157.2
Nov. 27, 2002 (GB) 0227649.1

(51) Int. Cl. A61B 5/05

(52) U.S. Cl. 600/425

(57) ABSTRACT

Apparatus for obtaining an image of a specimen (6) by optical projection tomography comprises a light scanner, such as a light-scanning confocal microscope (1, 2, 3) for subjecting the specimen (6) to a scanning movement of incident light.

### FIELD OF THE INVENTION

This invention relates to optical projection tomography.

### BACKGROUND TO THE INVENTION

Optical projection tomography is a technique for producing three-dimensional images of specimens, one example being disclosed in the applicant's specification WO 02/095476. The invention aims to provide a different way of directing the light onto the specimen, particularly in the case of fluorescent imaging, with a view to reducing noise or interference in the series of images and providing improved depth of focus in the series of images.

### SUMMARY OF THE INVENTION

According to one aspect of the invention there is provided apparatus for obtaining an image of a specimen by optical projection tomography, the apparatus comprising light-scanning means and a rotary stage for rotating the specimen to indexed positions in each of which the specimen is in use subjected to a scanning movement of incident light by the scanning means.

The incident light may be scanned in a direction perpendicular to an optical axis defined by the light passing through the apparatus.

The light scanning means may form part of a confocal scanning microscope.

According to another aspect of the invention there is provided a method of obtaining an image of a specimen by optical projection tomograpy, the method comprising scanning the specimen with a light beam and detecting light emanating from the specimen to derive the image.

Preferably, the detector detects light which exits or by-passes the specimen parallel to the beam incident on the specimen.

The incident light is preferably scanned in a raster pattern, one complete scan being undertaken at each indexed position of the specimen.

According to the present invention, samples for use in the present invention may be prepared as described in the earlier patent applications and/or employing conventional pathological and histological techniques and procedures well known to persons skilled in the art.

For example, in-situ hybridisation (particularly useful for detecting RNAs):Hammond K L, Hanson I M, Brown A G, Lettice L A, Hill R E “Mammalian and Drosophila dachsund genes are related to the Ski proto-oncongene and are expressed in eye and limb”. Mech Dev. 1998 June;74(1-2):121-31.

Immunohistochemistry (particularly useful for detecting proteins and other molecules): Sharpe J, Ahlgren U, Perry P, Hill B, Ross A, Hecksher-Sorensen J, Baldock R, Davidson D. “Optical projection tomography as a tool for 3D microscopy and gene expression studies” Science. 2002 Apr. 19;296(5567):541-5.

It will be appreciated that modification may be made to the invention without departing from the scope of the invention.

### BRIEF DESCRIPTION OF THE DRAWINGS

The invention will now be described, by way of example, with reference to the accompanying drawings, in which:

FIG. 1 is a diagram of the apparatus forming the preferred embodiment of the invention,

FIGS. 2a and 2b show how the microscope optics of the apparatus can be arranged to have low numerical aperture or high numerical aperture,

FIG. 3 shows known image-forming optics,

FIGS. 4 and 5 show the image-forming optics of an optical system of the inventive apparatus,

FIGS. 6a, 6b, 6c and 6d show representative light paths for the optical system of the inventive apparatus,

FIGS. 7a, 7b and 7c illustrate how different degrees of refraction affect operation of the optical system,

FIG. 8 illustrates how refraction is measured using a one-dimensional array of detectors, and

FIGS. 9 to 12 illustrate, in three dimensions, the operation of the optical system.

### DETAILED DESCRIPTION OF THE DRAWINGS

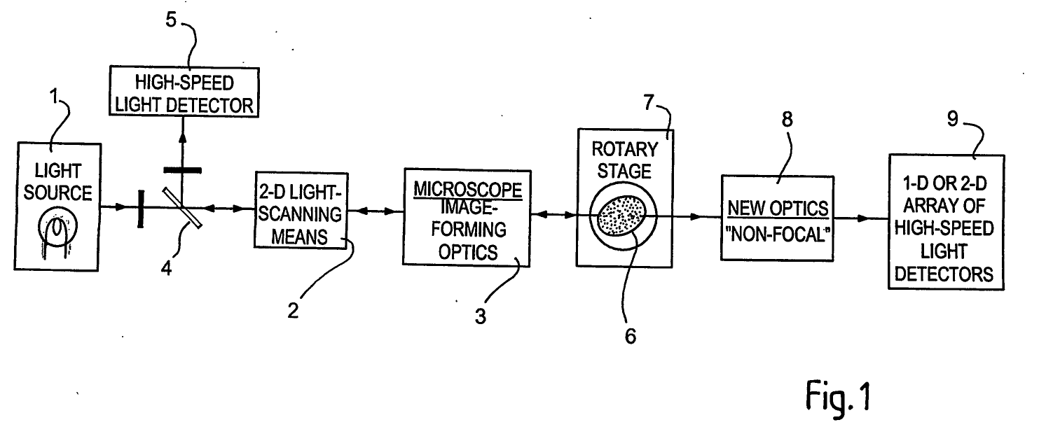

Referring to FIG. 1, the apparatus comprises a light source 1 (in the form of a laser) which supplies light to a two-dimensional light scanning means 2, the scanning mechanism of which has a dual mirror system. Light with a scanning motion is fed through image-forming optics 3. A dichroic mirror 4 interposed between the light source 1 and the scanning means 2 directs returned light to a high speed light detector 5. The components 1 to 5 may be provided by a confocal light-scanning microscope.

Light from the optics 3 passes through a specimen 6 which is rotated within, and supported by, a rotary stage 7 which in structure corresponds to the rotary stage disclosed in the applicant's co-pending International Patent Application No. PCT/GB02/02373. The rotary stage 7 rotates the specimen 6 to successive indexed positions at each of which one complete scan of the excitation light is undertaken whilst the specimen is stationary. After passing through the specimen 6, the light is processed by an optical system 8 which directs the light to a one-dimensional or two-dimensional array of high speed light detectors 9.

In fluorescence mode, light from the specimen 6 is returned through the optics 3 and the scanning means 2 and thence, via the mirror 4, to the high speed light detector 5. In this method of fluorescence imaging, the excitation light enters one side of the specimen and leaves the specimen from the same side thereof before being detected. It is in the transmission mode, to be described, that the components shown to the right of the stage 7 in FIG. 1 are used.

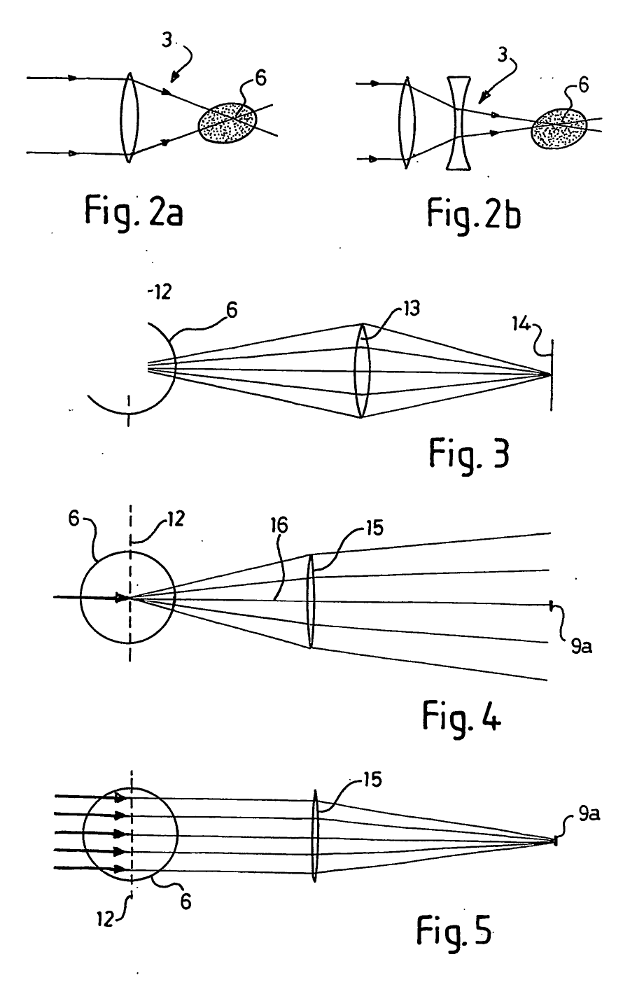

The microscope optics 3 may have a high numerical aperture (FIG. 2a) or may be adapted to have a low numerical aperture (FIG. 2b) which is useful for some specimens to be imaged.

FIG. 3 illustrates a known image-forming system. The light from any point on the focal plane 12 (within the specimen) is collected and refracted by a lens 13 towards a single point in the image plane 14. There exists a symmetry such that any point on the image plane 14 maps to a point in the focal plane 12 and vice versa.

By contrast, the need for an image-forming optical arrangement is removed in the inventive “non-focal” optics of FIGS. 4 and 5 which displays no such symmetry. The non-focal optical system 8 is represented by a convex lens 15. The light from a single point on the focal plane 12 is not focussed onto a single light detector. It is diverged such that only the light which exits or by-passes the specimen 6 parallel to the incident beam reaches the single light detector 9a positioned on the optical axis. The purpose of the lens 15 in FIGS. 4 and 5 is different from FIG. 3. It functions in a light-scanning situation. The light beam is scanned (e.g. in a raster pattern) across the specimen through a multitude of different positions (five of which are illustrated as the black arrows in FIG. 5). The purpose of the non-focal optical system 8 (i.e. the lens 15) is to direct onto the single light detector 9a, light which exits or by-passes the specimen parallel to the incident beam, irrespective of the scanning position of the light beam. In specimens which cause significant scattering of light the system allows a higher signal-to-noise ratio to be obtained by limiting detection of scattering light.

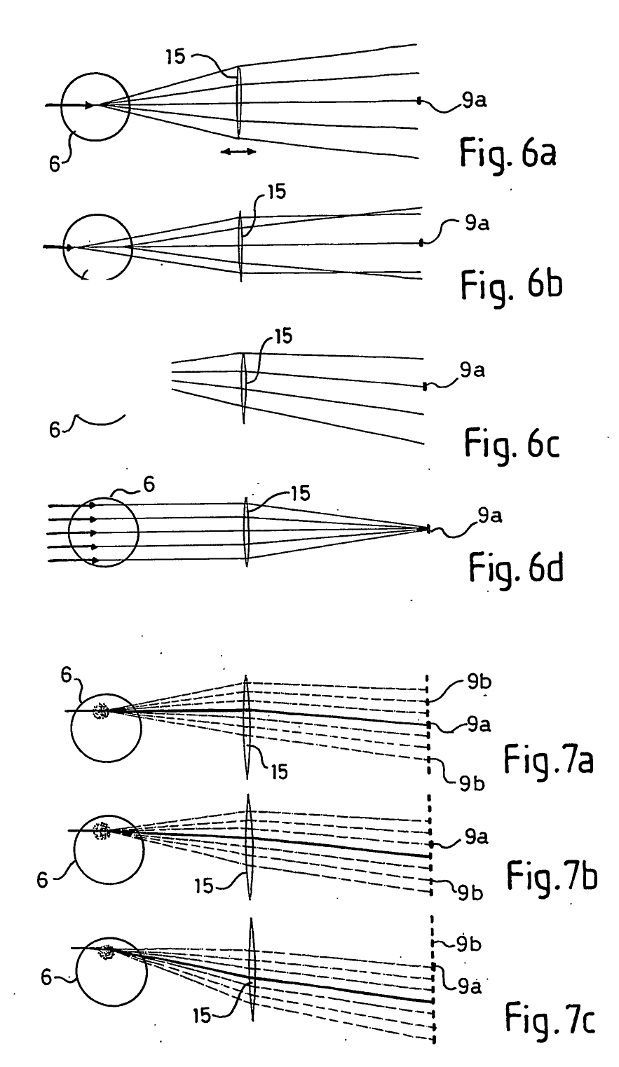

FIGS. 6a to 6d, which illustrate scattering as an example to show deviation from the original beam position, illustrate some representative light paths for rays (derived from a laser beam) emitted from the specimen 6 while passing through the non-focal optical system. The beam approaching the specimen from the left is the beam incident on the specimen.

In FIG. 6a rays scattered from a point in the centre of the specimen 6 are diverged away from the light detector 9a. The proportion of scattered rays which are detected can be adjusted by changing the effective size of the detector. An adjustable iris allows this control (which is very similar to the pin-hole in a scanning confocal microscope). Alternatively, the position of the lens can be adjusted to cause more or less divergence of the scattered rays. In optical image-forming systems, an airy disc is the interference pattern produced by the light emitted from a single point within the specimen. Optical systems which produce larger airy discs have lower resolving power, as airy discs from neighbouring points within the specimen will overlap. The concept of the airy disc is not strictly relevant to a projection-measuring system like this, however a similar concept does exist. In the case of the non-focal optics described here, light from each projection creates a very broad distribution of intensities (at the position of the detector) similar to a broad airy disc, which might suggest low resolving power. However, as only a single projection is measured at any one time even very broad distributions cannot interfere with each other.

In FIG. 6b rays scattered from other points along the same line sampled in FIG. 6a, are also diverged away from the light detector 9a.

In FIG. 6c unscattered light from a different scanned position (black arrow) is emitted from the specimen 6 substantially parallel to the optical axis, and is therefore refracted towards the light detector 9a. As in FIGS. 6a and 6b, scattered light is directed away from the detector 9a.

In FIG. 6d unscattered rays from any scanned position are directed onto the light detector 6. The arrows represent successive positions of the laser beam as it is scanned across the specimen 6 in a direction perpendicular to the optical axis.

All experiments done so far with optical projection tomography have had to assume that although some of the light is scattered, the refractive index of the specimen is uniform. Recent experiments have demonstrated that a number of important specimens (including medical imaging of biopsies) display non-uniform refractive indexes. This means that the current algorithms are not accurately imaging the specimen—distortions and artefacts are introduced. The apparatus described reduces this problem by measuring information not previously available relating to the angle at which a light beam exits from the specimen. In general, in specimens with low scattering but non-uniform distribution of refractive index the system allows this non-uniform distribution to be calculated by measuring the degree of refraction experienced by each projection.

In the use of the present apparatus a clearing agent (such as BABB) is used such that the majority of the light is not scattered. It is however subject to a different form of disruption—refraction. In FIG. 7, scattered light is indicated by broken lines, while the main path of light is shown as a solid line. In the first example of FIG. 7a this path is not bent as it passes through the specimen 6 (it is only refracted on passing through the lens). The main path does pass through a region of the specimen with a higher refractive index than the rest (grey disc), however both the interfaces it encounters between regions of differing refractive index are perpendicular to the light path, so no refraction occurs.

In the second case of FIG. 7b, the illumination beam is slightly higher and therefore the interfaces it encounters between the grey region and the white region of the specimen (different refractive indexes) are slightly displaced from perpendicular. This causes two slight refractions of the main path such that when the light emerges from the specimen it is no longer parallel to the incident beam and is directed slightly to the side of the original central light detector 9a. If auxiliary light detectors 9b are positioned on either side of the central detector 9a, these can measure the degree of refraction. Any projection will give a certain distribution of intensities along the array of light detectors. The distribution of intensities can be used to determine the angle at which the main light path emerged from the specimen. The system need only determine where the centre of this distribution is (usually the strongest intensity) to measure the angle at which the main light path emerged from the specimen. In the last case of FIG. 7c, a different scanned position has caused greater refraction of the beam, which is reflected in a further shift along the array of detectors.

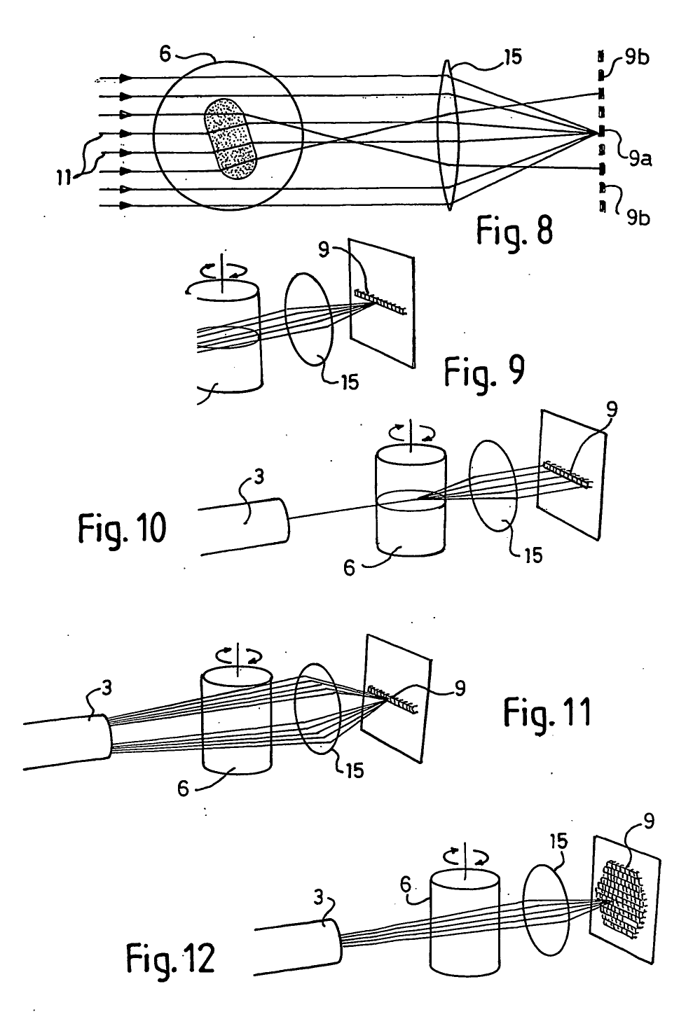

In FIG. 8, an oblong region of the specimen 6 has a higher refractive index (grey shape) than the rest. Rays passing around the specimen are not refracted and so are directed to the central light detector 9a. Rays passing through the middle of the specimen (middle two rays 11 in FIG. 8) are refracted twice. The two interfaces which the light passes through (white-to-grey and then grey-to-white) are parallel with each other, and the light rays therefore exit the specimen at the same angle that they entered it. These rays are also directed onto the central detector 9a. Rays passing through other parts of the grey region are also refracted twice but do not pass through parallel interfaces, so these rays are detected by the adjacent light detectors 9b.

The fact that some rays will be refracted and still exit the specimen 6 parallel to the incident beam is not a problem. The example of FIG. 8 shows only one of the many sets of projections taken through this section. Full imaging involves capturing such a data set for many orientations through the section, and the combination of all this data allows a full reconstruction of the distribution.

FIGS. 9 to 12 show three-dimensional views of the apparatus. In FIG. 9, all un-refracted (and unscattered) rays through a two-dimensional section of the specimen are focused onto the central light detector of the array. The specimen 6 is rotated about a vertical axis between indexed positions in each of which a complete scan is undertaken.

FIG. 10 shows the path of scattered or refracted light onto auxiliary light detectors.

FIG. 11 illustrates that the lens (or optical system) allows the one-dimensional array of detectors 9 to capture data from a full two-dimensional raster-scan of the specimen. A row of scanned positions is always directed down or up to the row of detectors, irrespective of the vertical height of the scan.

A two-dimensional array of light detectors 9 may be used instead of a one-dimensional array, as shown in FIG. 12. This would be able to measure light which is scattered or refracted above or below the plane occupied by the light rays shown in FIG. 12.

In prior-art wide-field optical projection tomography, each pixel of the CCD should record the information from an approximate projection through the specimen. Wide-field fluorescence optical projection tomography suffers a problem due to the fact that illumination/excitation of the specimen must also be wide-field. If the optical properties of the specimen cause internal scattering of light, then many photons exit the specimen along trajectories which cause them to be detected by pixels which do not represent the projection from which the photon originated. This adds significant noise to the image. The light-scanning invention described here avoids this problem because only the fluorescent particles within the approximate projection are excited at any one time.

The data derived from the detector array 9 optics is interpreted by an algorithm.

Many different algorithmic approaches already exist for performing back-projection calculations. One approach is to use a standard linear filtered back-projection algorithm (as in U.S. Pat. No. 5,680,484). Other approaches include iterative, maximum entropy and algebraic reconstruction technique. (R. Gordon et al., “Three-Dimensional Reconstruction form Projections: A Review of Algorithms”.

The algorithm works as follows:

1. The data is used as if it were parallel (or fan-beam) data to perform back-projection. This produces a “fuzzy” estimation of the distribution of absorption characteristics of the specimen, or alternatively a fuzzy distribution of the fluorescence of the specimen.

2. A first approximation of the distribution of refractive index is estimated. This can be done in a number of ways. One useful method is to assume that the absorption or fluorescent distribution will reflect the distribution of refractive index. Within each section a 2-D gradient vector is calculated for each voxel. An alternative is to start with a uniform or a random distribution.

3. The estimated refraction distribution is used to perform a forward-projection, i.e. a prediction of what the projection data should look like if the initial estimate of the refraction distribution was correct.

4. The predicted projections and the actual projections are compared.

5. The estimated refraction distribution is modified. The projections with a greater difference between predicted and actual, pin-point which regions of the distribution need more modification. For example, in the case of the grey shape shown in FIG. 8, projections from the curved ends of the oblong will differ greatly from the predictions due to the large amount of refraction. Voxels in the regions therefore have their predicted refraction indexes changed more than other regions.

6. The loop from 3 to 6 is repeated until no further improvements to the predicted projections can be made.

The algorithm approach above can also be used to interpret other optical signals, for example fluorescence or scattering.

It will be appreciated that modification may be made to the invention without departing from the scope of the invention.

1. Apparatus for obtaining an image of a specimen by optical projection tomography, the apparatus comprising light scanning means and a rotary stage for rotating the specimen to indexed positions in each of which the specimen is in use subjected to a scanning movement of incident light by the scanning means.

2. Apparatus according to claim 1, wherein the incident light is scanned in a direction perpendicular to an optical axis followed by the light passing through the apparatus.

3. Apparatus according to claim 1, wherein the incident light is scanned in a raster pattern, one complete scan being undertaken at each indexed position of the specimen.

4. Apparatus according to claim 1, wherein the light scanning means form part of a confocal scanning microscope.

5. A method of obtaining an image of a specimen by optical projection tomography, comprising scanning the specimen with a light beam and detecting light emanating from the specimen to derive the image.

6. A method according to claim 5, wherein the light passes through the specimen prior to being detected.

7. A method according to claim 5, wherein the light enters from one side of the specimen and leaves the specimen from the same side thereof.

8. A method according to claim 5, wherein the specimen is rotated to indexed positions and one complete scan is undertaken at each indexed position of the specimen.

9. A method according to claim 5, wherein the detector detects light which exits or by-passes the specimen parallel to the beam incident on the specimen.

10. A method according to claim 5, wherein the light is laser light.

11-12. (canceled)
